(19) United States
(12) Patent Application Publication
(10) Pub. No.: US 2015/0139940 A1
(43) Pub. Date: May 21, 2015

(54) STRESS-REGULATED PROKARYOTIC EXPRESSION CASSETTES

(71) Applicant: INSTITUT NATIONAL DE LA RECHERCHE AGRONOMIQUE (INRA), Paris Cedex 07 (JP)

(72) Inventors: Luis Bermudez Humaran, Jouy En Josas (FR); Philippe Langella, Velizy-Villacoublay (FR); Pascale Kharrat, Fontenay-aux-Roses (FR)

(21) Appl. No.: 14/402,282

(22) PCT Filed: May 15, 2013
(86) PCT No.: PCT/IB2013/053983

§ 371 Date: Nov. 19, 2014

(30) Foreign Application Priority Data
May 21, 2012 (FR) 1254613

(51) Int. Cl. C12N 15/74; A61K 38/20; C12N 7/00; A61K 39/12

(52) U.S. Cl. CPC C12N 15/746; A61K 39/12; A61K 38/2066; C12N 7/00; A61K 48/00
USPC 424/85.2; 435/320.1; 435/252.3; 424/200.1

(57) ABSTRACT

The invention relates to prokaryotic expression cassettes comprising a stress-regulated promoter, the promoter of the GroESL operon of Lactococcus lactis.

Said cassettes can be used for the expression of genes of interest in gram-positive bacteria, and especially for producing and releasing a protein of interest when the bacteria are administered to an individual.

The invention relates to the production of heterologous proteins in Gram-positive bacteria, specifically lactic acid bacteria.

In addition to their traditional uses in the food processing industry, which have proven their safety, lactic acid bacteria, especially Lactococcus and Lactobacilli, are currently used increasingly as host cells for the production of heterologous proteins. Some recombinant lactic acid bacteria are used as living vectors to deliver, via the mucosal pathway, molecules that are beneficial to health (journal articles: Daniel et al., Trends Biotechnol., 29(10), 409-508, 2011; and Bermùdez-Humaràn et al., Microb. Cell Fact., 10 (Suppl 1): S4, 2011).

Among the lactic acid bacteria, Lactococcus lactis in particular is a non-invasive and non-pathogenic bacterium, which is widely used in the making of dairy products, cheeses especially. It is 90% resistant to gastric passage in humans and in mice. L. lactis is the model lactic acid bacteria and, along with Escherichia coli and Bacillus subtilis, is one of the best-described bacteria. For this reason, L. lactis is considered an excellent candidate for the expression of proteins of interest with regard to health of the mucous membranes (antigens, cytokines, hormones, etc.).

Steidler et al. (Science, 289, 1352-1355, 2000) describe the use of a strain of recombinant L. lactis, e.g., that expresses the interleukin-10 (IL-10) cytokine under the control of the constituent promoter P1 of L. lactis.

The same team subsequently attempted to identify strong constituent promoters that could be used to express proteins of therapeutic interest in bacteria of the genus Lactococcus (PCT Application WO2008/084115), these promoters being defined as being stronger than the promoter of the gene of thymidylate synthase (thyA) of L. lactis. 53 candidate promoters that potentially meet this definition have been identified by transcriptome and proteome analyses. Of these, 17 have been subcloned to test their capacity to control the expression of a heterologous protein: this includes promoters of the genes rpoB, dpsA, glnA, glnR, pepV, atpD, pgk, fabF, fabG, rpoA, pepQ, rpsD, sodA, luxS, rpsK, rpIL, and hIIA (named as such in L. lactis MG1363). Four of these promoters have in fact achieved the desired expression level: the promoters of the genes dpsA, pepV, sodA, and hIIA.

The promoters described in the documents cited above are all constituent promoters. In certain cases, it is preferable to use inducible promoters that allow expression to be initiated or stopped at the desired moment. Indeed, certain proteins, such as cytokines, may accumulate inside the cytoplasm of the bacteria and degrade the host cell. Expression systems that integrate an inducible promoter enable better control of the expression of the heterologous protein and make it possible to prevent the negative effects that may result from its overproduction.

Although a very large number of genes whose expression is regulated by various factors is known among lactic acid bacteria and in L. lactis in particular, at present there is only a limited choice of inducible promoters that can be used in actual practice for building expression cassettes of genes of interest (journal articles: De Vos, Curr. Op. Microbiol., 2, 289-295, 1999; Morello et al., J. Mol. Microbiol. Biotechnol., 14(1-3); 48-58, 2008; Nouaille et al., Genet. Mol. Res., 2(1): 102-111, 2003). Indeed, this use requires not only that the promoters in question be inducible, but that there also be a sufficient differential between the various states of induction; ideally, expression must reach an elevated level under induction conditions, and be absent or low under non-induction conditions.

The NICE (“NIsin Controlled Expression System”) system (Kuipers et al., J. of Biol. Chem., 270 (45): 27299-27304, 1995; de Ruyter et al., Appl. Environ. Microbiol., 62: 3662-3667, 1996; Kuipers et al., J. of Biotechnol., 64(1): 15-21, 1998; Mierau et al., Appl. Microbiol. Biotechnol., 68(6): 705-17, 2005) is currently the most widely used system for expressing a gene of interest based on whether a bacteriocin, nisin, is present within or absent from the external environment. However, the disadvantages inherent in this system are, on the one hand, the need for the presence of regulator genes of this system (brought in by plasmids, or cloned and integrated into the bacterial chromosome), and, on the other hand, the need for a stage consisting of placing nisin in the presence of a bacteria.

In this context, the inventors have attempted to implement an inducible expression system that is suitable for the expression of heterologous proteins of interest within lactic acid bacteria that are to be used as a therapeutic or vaccine vector; they developed the idea of researching an expression system that would be induced the bacteria were being administered to the subject to be treated. They started with the hypothesis that this administration, which places the bacteria under conditions that are quite different from their traditional living conditions, would be able to induce in the latter various types of stress: this may include heat stress, if the body temperature of the subject to be treated is sufficiently high relative to the optimal growth temperature of the administered bacteria; in the case of oral administration, heat stress may be accompanied by acid stress during passage in the stomach (where the pH level is about 1.5 to 2), and then by biliary stress in the duodenum.

The inventors attempted to discover whether it was possible to identify, among the promoters of many genes identified as being induced by one or several of these stresses (for review purposes see, e.g., Van de Guchte et al., Antonie van Leeuwenhoek, 82, 187-216, 2002), a promoter that can in fact be used in actual practice to control the inducible expression of heterologous proteins.

They focused on the promoter of the GroESL operon of Lactococcus lactis. The GroES and GroEL proteins, which are present in many bacteria, are chaperones and heat-shock proteins (hsp). The GroESL operon of Lactococcus lactis subsp. lactis was described by Kim et al., (Gene, 127(1): 121-126, 1993), who reported that its transcription was inducible by heat shock. Arnau et al. (Microbiology, 142, 1685-1691, 1996) studied the induction kinetics of the transcription of the dnaK, dnaJ, and groEL genes of Lactococcus lactis subsp. cremoris during heat shock caused by raising the temperature from 30° C. (optimal growth temperature of Lactococcus lactis) to 43° C. They observed a level of transcription ranging from 10 times (for dnaJ and groEL) to 100 times (for dnaK) the base level for the 15 first minutes of heat shock, and decreasing after 20 minutes.

The inventors cloned the promoter of the GroESL operon of Lactococcus lactis, and tested its ability to control the expression of a heterologous protein and its inducibility under conditions reproducing those of in vivo administration in humans.

They found that when base conditions prevailed (that is, during optimal growth conditions for L. lactis), the PGroESL promoter allowed the expression of a heterologous protein, at a low level, and that raising the temperature to 37° C., although lower than the temperatures (over 40° C.) traditionally used for inducing heat stress in L. lactis, was sufficient to increase by 1.3 to 1.5 times the amount of heterologous protein produced.

Therefore, the goal of the present invention is an expression cassette that includes:
The promoter (PGroESL) of Lactococcus lactis, and
A heterologous nucleotide sequence that codes for a polypeptide of interest, placed under the transcriptional control of said promoter.

We employ the following definitions here: “PGroESL promoter of L. lactis” is a polypeptide of 82 to 150 bp comprising a functional promoter in L. lactis, and including a nucleotide sequence that has at least 90%, and in order of increasing preference, at least 91%, at least 92%, at least 93%, at least 94%, at least 95%, at least 96%, at least 97%, at least 98%, at least 99%, or 100%, identity with the following sequence:

(SEQ ID NO.: 1)
CTTGACTAAATCTGACCATTGAGATAAAATAAGAATATGTTAGCACTCAA
CTATTAAGAGTGCTAAAAATAAAAAATGGAGG.

The PGroESL promoter contains the following elements of cis-regulation, positioned by way of example on SEQ ID NO.: 1:
A binding site for the CtsR regulatory protein, at positions 1-17, the CtsR protein being involved in the regulation of various genes that intervene in the response to heat shock;
A CIRCE element (for “Controlling Inverted Repeat of Chaperone Expression”), at positions 40-66, which is an inverted repeated sequence involved in regulating the expression of chaperone proteins;
A −35 box, at positions 41-46;
A −10 box, at positions 64-69;
An ACD (or ACiD) box (see Madsen et al., Molec. Microbiol., 56(3), 735-746, 2005), at positions 29-42, involved in the response to the acid pH; and
A ribosome binding site (RBS), at positions 78-82.

The SEQ ID NO.: 1 sequence, listed here as a reference sequence, comes from the model strain of Lactococcus lactis subsp. cremoris MG1363 (GenBank NC_{—}009004).

Other PGroESL promoters that can be used for the construction of an expression cassette in accordance with the invention can also be isolated and cloned from any strain of L. lactis by using standard genetic engineering techniques.

As non-limiting examples, we cite:
The PGroESL promoter of the A76 strain (GenBank CP003132) of Lactococcus lactis subsp. cremoris, which includes the SEQ ID NO.: 1 sequence;
The PGroESL promoter of the NZ9000 strain (GenBank CP002094) of Lactococcus lactis subsp. cremoris, which includes the SEQ ID NO.: 1 sequence;
The PGroESL promoter of the SK11 strain (GenBank NC_{—}008527) of Lactococcus lactis subsp. cremoris, which includes the following sequence:

(SEQ ID NO.: 2)
CTTGACTAAATCTGACCATTGAGATAAAATAAGAATATGTTAGCACACAA
CTATTAAGAGTGCTAAAAATAAAAAATGGAGT;

The PGroESL promoters of the IL1403 (GenBank NC_{—}002662) or CV56 (GenBank CP002365) strains of Lactococcus lactis subsp. lactis, which include the sequence:

(SEQ ID NO.: 3)
CTTGACTAAATCTGACCATTGAGATAAAATAAGAATATGTTAGCACTCGT
TTAATAAGAGTGCTAAAAAATAAAAAATGGAGG;

The PGroESL promoter of the KF147 strain (GenBank NC_{—}013656) of Lactococcus lactis subsp. lactis, which includes the following sequence:

(SEQ ID NO.: 4)
CTTGACTAAATCTGACCATTGAGATAAAATAAGAATATGTTAGCACTCGT
TTAACAAGAGTGCTAAAAAATAAAAAATGGAGG.

We define here as a “heterologous nucleotide sequence” any nucleotide sequence other than the one naturally located in L. lactis downstream of the PGroESL promoter, and especially any nucleotide sequence that does not include those that code for the GroES and GroEL proteins of L. lactis. Preferably, this will be a sequence that does not originate from L. lactis.

It may involve any sequence that codes for a polypeptide of interest, especially one of therapeutic or vaccination interest, that one wishes to have the host bacteria express. Said polypeptide may, if desired, be a chimeric polypeptide, combining polypeptide sequences of various origins.

By way of non-limiting examples, said heterologous nucleotide sequence may be:
A nucleotide sequence that codes for a cytokine, e.g., leukin-10 (IL-10), IL-12, IL-2, IL-4, GM-CSF (“Granulocyte-Macrophage Colony-Stimulating Factor”), or TGF-β (“Transforming growth factor-beta”);
A nucleotide sequence that codes for a vaccine antigen, of viral or bacterial origin, or of eukaryote origin, such as a fungal antigen, or a parasitic antigen; the antigen is, e.g., one of the proteins E6, E7, or L1 of human papilloma virus (HPV) Type 16, the VapA protein of Rhodococcus equi, the L7/L12 protein of Brucella abortus, or the Nuc protein of Staphylococcus aureus;
A nucleotide sequence that codes for a protease inhibitor, e.g., Elafin (or Trappin-2), SLPI (Secretory Leukocyte Protease Inhibitor), or one of the serine protease inhibitors (serpin family);
A nucleotide sequence that codes for a hormone, e.g., leptin, insulin, human growth hormone, IGF-1 (Insulin-like Growth Factor), IGF-2, or Epidermal Growth Factor (EGF);
A nucleotide sequence that codes for an enzyme of bacterial or eukaryote origin, e.g., a catalase, a superoxide dismutase, a lipase, a polymerase, an isomerase, or a ligase.

An expression cassette according to the invention may, if desired, additionally include the elements needed to enable addressing of the protein of interest on the cell surface, or its secretion into the external medium.

In this framework, the expression cassette according to the invention may include a nucleotide sequence that codes for an extracellular addressing peptide placed under the transcriptional control of the PGroESL promoter, said expression cassette enabling cloning of the heterologous nucleotide sequence of interest in translational fusion with said addressing peptide. The addressing peptide may be, e.g., a secretion signal peptide, a transmembrane domain, a cell-wall-anchoring signal, etc.

Many addressing peptides that can be used as part of the present invention are known. As nonlimiting examples, we cite the signal peptide of the Exp4 protein of L. lactis, defined by the sequence:

(SEQ ID NO.: 5)
MKKINLALLTLATLMGVSSTAVVFA

or the peptides identified in the publications of Poquet et al. (J. Bacteriol., 180, 1904-1912, 1998), of Le Loir et al. (Appl. Environ. Microbiol., 67, 4119-4127, 2001), and by Ravn et al. (Gene, 242, 347-356, 2000), respectively.

The invention also relates to a recombinant vector that includes an insert comprised of an expression cassette according to the invention.

Recombinant vectors in accordance with the invention can be obtained, in the standard way, by inserting an expression cassette according to the invention into a functional vector in L. lactis.

A large number of functional vectors in L. lactis are known. A person skilled in the art will select the most appropriate vector (integrative or extrachromosomal vector, vector with a varying number of copies) depending upon the nature of the heterologous protein to be expressed, and the particular use for which it is intended.

Another goal of the invention is a bacterium of the species Lactococcus lactis, transformed by at least one expression cassette according to the invention.

If desired, this may be a bacterium originating from a strain of L. lactis that includes one or more modifications of its genome. This may include, e.g., modifications aimed at improving the production and/or the secretion of proteins expressed in said bacterium, and/or at preventing their degradation. For example, in the context of producing exported proteins, one may use a bacterial strain in which PrtP protease activity and/or in cases where HtrA and/or C1pP protease activity are inactive, such as that described in PCT Application WO00/39309, or a bacterial strain that overproduces a protein that stabilizes exported proteins, such as the Nlp4 protein of Lactococcus lactis, or one of its homologs (Poquet et al. 1998, publication cited above), or a bacterial strain modified by the addition of a heterologous gene responsible for protein secretion, such as the secDF gene of Bacillus subtilis (Nouaille et al., Appl. Environ. Microbiol., 72(3): 2272-9, 2006).

One may also use a bacterium that includes a defective auxotrophic gene in order to make survival of this bacterium dependent on the presence of one or more specific compounds. The intended auxotrophic gene may be the thyA gene (PCT Application WO2011/086172), which codes for thymidylate synthase, an enzyme that generates thymidine monophosphate (dTMP), which is then phosphorylated into thymidine triphosphate, involved in DNA synthesis and repair, or the alr gene, which codes for alanine racemase (Bron et al., Environ. Microbiol., 5663-5370, 2002). The selected auxotrophic gene may be inactivated by any known method, e.g., by inserting the heterologous nucleotide sequence at the locus of this gene, in the bacterial chromosome.

Another goal of the present invention is the use of a bacterium transformed according to the invention in order to produce a polypeptide of interest when said bacterium is subjected to heat shock, which results from its exposure to a temperature higher than its normal growth temperature.

In this framework, the bacteria transformed according to the invention, if desired, may be used to produce in vitro a polypeptide of interest. Preferably, they may be used specifically as medications or vaccines, as vectors for administering a protein of therapeutic or vaccine interest.

In particular, the present invention includes as a goal a method for causing a polypeptide of interest to be produced by a bacterium transformed according to the invention that contains a sequence coding for said polypeptide of interest placed under the transcriptional control of the PGroESL promoter, characterized in that said bacterium is exposed to a temperature higher than 36° C. and lower than or equal to 40° C. in order to induce the expression of said polypeptide. Preferably, said temperature will be equal to or higher than 36.5°, but it is especially preferred that it be equal to or higher than 37° C.

In a preferred implementation of this method, said polypeptide is a therapeutic or vaccine protein of interest, and exposure of said bacterium to the temperature that enables the expression of said polypeptide to be induced is carried out by administering said bacterium to a subject, who preferably is a human being, or an animal if desired, specifically a mammal, whose body temperature is higher than 37° C.

The bacteria according to the invention may be administered in particular via the mucous membranes, more particularly intranasally, orally, rectally, or vaginally, and preferably intranasally or orally. Their use enables in situ expression of the protein of interest in the relevant mucous membranes.

The use of bacteria transformed according to the invention is particularly interesting in the context of treating diseases such as chronic inflammatory bowel disease (IBD), because it makes it possible to deliver locally the protein of interest, directly into the intestine, during passage of the bacteria in the duodenum, thus enabling a highly localized action of the protein and therefore greater efficacy. In this framework, they may be used for the administration of anti-inflammatory cytokines, such as interleukin-10.

As other non-limiting examples of the use of bacteria transformed according to the invention, we note:
Vaccination against cervical cancer, via intranasal administration of the E7 antigen of Type 16 human papillomavirus;
Therapy against chronic inflammatory bowel disease (IBD), via oral administration of anti-inflammatory molecules such as cytokine IL-10 or Elafin;
Therapy against allergies, via intranasal administration of pro-inflammatory molecules such as cytokine IL-12 or TGF-β.

As a guideline, the bacteria transformed according to the invention can be administered at a dose of between 1×10⁸ and 1×10¹⁰ CFU (colony-forming units), preferably between 5×10⁸ and 1×10¹⁰ CFU, advantageously between 5×10⁸ and 5×10⁹ CFU for intranasal administration, and between 1×10⁹ and 1×10¹⁰ CFU for oral administration. For example, they can be administered at a dose of 1×10⁹ CFU intranasally or 5×10⁹ CFU orally.

### EXAMPLE 1

### Cloning of the Groesl Promoter and Construction of the Stress-Regulated Expression System

A DNA region upstream of the GroESL gene was amplified by PCR starting with genomic DNA from the Lactococcus lactis MG1363 strain.

The primers used are the BglII-ProGroEL sense primer of the CCAAGATCTAAATGTTTTCTCTTGACTAAATCTGACC sequence (SEQ ID NO.: 6) and the NheI-ProGroEL antisense primer of the AGCTAGCGTTAATAAAGCAAGGTTTATTTTTTTCATATAC sequence (SEQ ID NO.: 7). These primers were designed so as to clone, thanks to the BG1II and NheI restriction sites (underlined), the PCR product in the pLB141 vector (Morello et al., 2008, cited above). Moreover, the antisense primer was selected in order to introduce the PCR product into this vector in read phase with the sequence coding for the 12 first amino acids of the PSExp4 signal peptide.

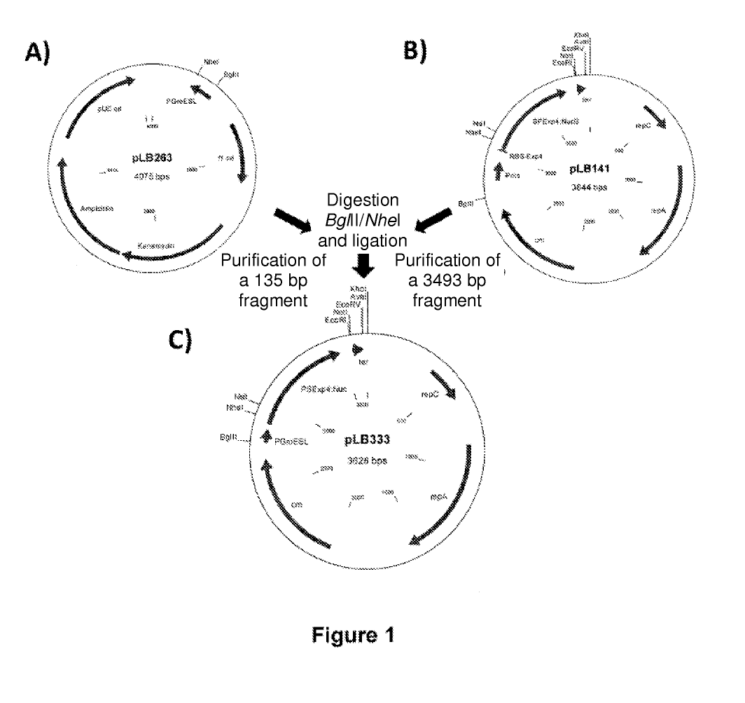

The PCR product was digested, purified, and cloned in the intermediate vector pCR®2.1 (TOPO®, Invitrogen). The resulting plasmid sequence, pLB263 (pCR:TOPO:PGroESL) (FIG. 1A), was confirmed by enzymatic digestion and by sequencing (Eurofins).

The amplified fragment, which contains the PGroESL promoter, has the following sequence:

(SEQ ID NO.: 8 )
ATCTAAATGTTTTCTCTTGACTAAATCTGACCATTGAGATAAAATAAGAA
TATGTTAGCACTCAACTATTAAGAGTGCTAAAAATAAAAAATGGAGGAAA
GTATA.

The PGroESL promoter was isolated from the pLB263 plasmid by digestion with the Bg1II and NheI enzymes, and cloned into the pLB141 vector (FIG. 1B), previously digested with the same enzymes, by replacing the promoter (P_{nisA}) initially present in this vector.

The resulting plasmid, pLB333 (pPGroESL:PSExp4:Nuc) (FIG. 1C), contains an expression cassette that expresses PSExp4 in fusion with the mature form of a model secreted protein, the nuclease of Staphylococcus aureus (Nus), under the control of the PGroESL promoter.

The pLB333 plasmid has a restriction site NsiI, between the sequence coding for PSExp4 and the reporter gene nuc coding for the Nuc nuclease, and a multiple cloning site at the end of the nuc gene and before the transcription terminator trpA (ter), to allow replacement of this gene by a gene of interest.

This plasmid was next introduced into L. lactis MG1363 to obtain the MG(pLB333) recombinant strain.

### EXAMPLE 2

### In Vitro Expression of the Nuc Protein

Expression of the nuc reporter gene was tested by Western blot technique after having induced heat shock in the MG(pLB333) recombinant strain obtained in Example 1.

To do this, an overnight preculture of the MG(pLB333) strain was diluted to 1/100^{th} in the morning, up to an optical density (OD) ranging from 0.4 to 0.6. Next, three volumes of 6 ml of the diluted preculture were poured into three tubes. In a parallel operation, three tubes of 6 ml of GM17 culture medium containing 10 μg/ml of chloramphenicol (Cm) were prepared and incubated at 30° C. and 37° C., respectively. Each of the three cultures was centrifuged for 5 minutes at 4700 rpm, then taken up again in the 6 ml of fresh, previously-heated GM17+Cm medium. The tubes obtained in this way were incubated for 5 and 30 minutes at 30° C. and 37° C.

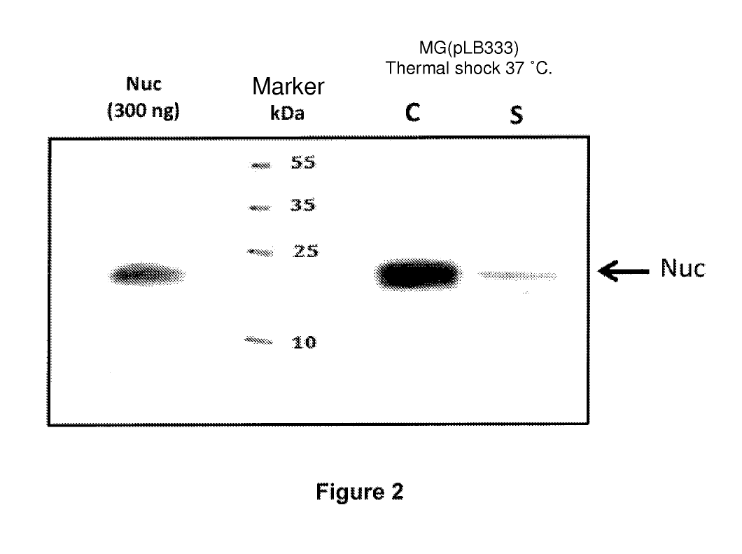

Next, the tubes were centrifuged for 10 minutes at 13000 rpm, and the pellet and supernatant were separated. The supernatant was precipitated in 10% TCA and this was repeated in some NaOH+blue (to obtain the S fraction). The pellet was placed in 150 μl of PBS+antiprotease 1X, a volume of beads was added to it, and the entire mass was passed through the FastPrep® system in order to destroy the bacteria and release the cytoplasm proteins (to obtain the C fraction). The production and secretion of the Nuc protein was analyzed by the Western blot technique with the help of anti-Nuc antibodies. The results, shown in FIG. 2, show that after the heat shock (5 minutes at 37° C.), the MG(pLB333) strain is capable of producing the Nuc protein in large amounts.

### EXAMPLE 3

### In Vitro Production of Interleukin-10

Interleukin-10 (IL-10) is an anti-inflammatory cytokine previously proposed for its use in the treatment of chronic inflammatory bowel disease (IBD).

The gene that codes for murine IL-10 was inserted into the pLB333 vector by replacing the nuc gene. The resulting plasmid, pLB350 (pPGroESL:PSExp4:IL-10), was introduced into L. lactis MG1363 to obtain the MG(pLB350) recombinant strain.

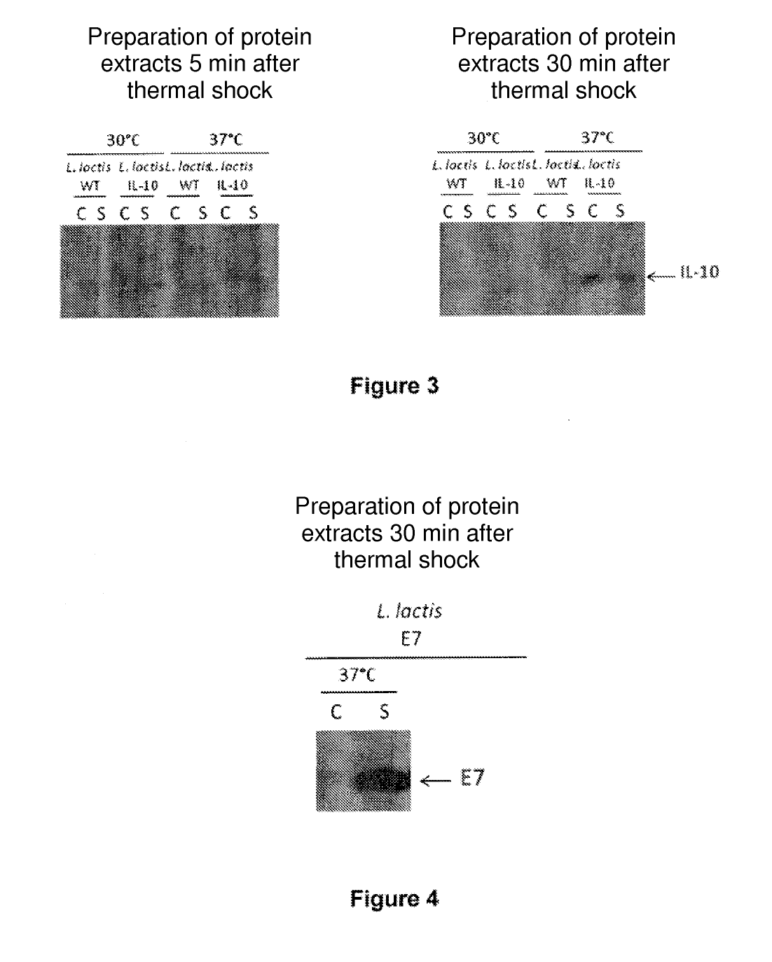

Based on the same protocol as the one described in Example 2, the production of IL-10 was confirmed by Western blot technique after having induced thermal shock in the MG(pLB350) recombinant strain. The results, shown in FIG. 3, demonstrate that following heat shock, the MG(pLB350) strain is capable of producing the protein IL-10 in large amounts.

### EXAMPLE 4

### In Vitro Production of the E7 Antigen of HPV Type 16

Human papillomavirus Type 16 (HPV-16) is one of the principal viruses responsible for cervical cancer. The use of E7 antigen of HPV-16, a protein found consistently in carcinomas caused by HPV infection, has already been proposed as a prophylactic or therapeutic vaccine against cervical cancer (Bermudez et al., J. of Med. Microbiol., 53, 427-433, 2004).

The gene coding for the E7 antigen was inserted into the vector pLB333 by replacing the nuc gene. The resulting plasmid, pLB356 (pPGroEL:PSExp4:E7), was introduced into L. lactis MG1363 to obtain the MG(pLB356) recombinant strain.

Using the same protocol as the one described in Example 2, the production of E7 antigen was confirmed by Western blot after having induced heat shock in the MG(pLB356) recombinant strain. The results, shown in FIG. 4, demonstrate that after the heat shock (30 minutes at 37° C.), the MG(pLB350) strain is capable of producing the E7 protein in large amounts and of secreting it efficaciously.

### EXAMPLE 5

### Use of Recombinant Bacteria that Produce E7 Antigen for Immunizing Mice

The effect of intranasal administration of the MG(pLB356) recombinant strain obtained in Example 4 was tested in a model of mice that develop cancerous tumors induced by a tumor cell line (TC-1) that expresses the E7 antigen of HPV-16.

Groups of 12 mice, 6 to 8 weeks old, were immunized intranasally with some PBS, or with the MG1363 (MG) wild strain, or with the MG(pLB356) recombinant strain.

To do this, a total of 1×10⁹ CFU of each strain were resuspended in 10 μL of PBS. It was determined that 1×10⁹ CFU of recombinant bacteria contained approximately 15 μg of E7 antigen. 5 μL of the suspension thus obtained were administered with the help of a micropipette into each nostril of the mice, on days 0, 14, and 28. On Day 35, or 7 days after the last immunization, half of each batch of mice (n=6) was sacrificed for immune response analysis, and a TC-1 tumor line was administered to the other half (n=6) by subcutaneous injection.

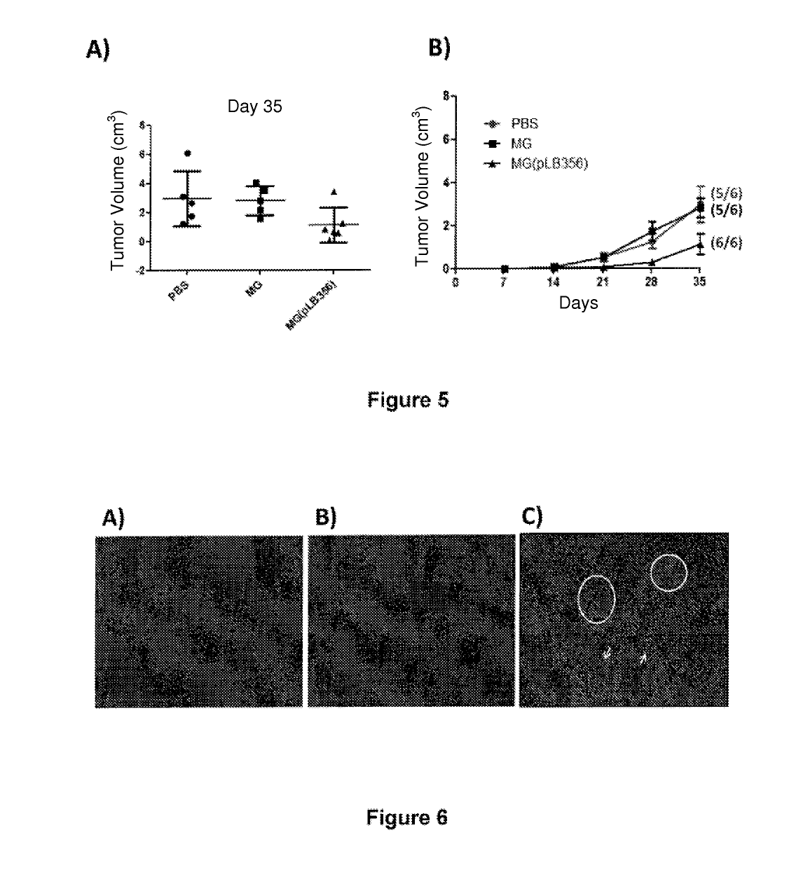

The volume of the tumors that developed in these mice was measured once a week for 5 weeks. The measurement results are shown in FIG. 5. The volume of the tumors in the mice vaccinated with the MG(pLB356) strain is smaller by 1 cm³ on average than that of the tumors that developed in the mice vaccinated with the MG control strain or with PBS, which is 2.7 cm³ and 2.9 cm³, respectively. The tumor volume is therefore significantly reduced in the mice vaccinated with the MG(pLB356) recombinant strain.

In addition, in the mice vaccinated with the MG(pLB356) strain, no mortality occurred (the 6 mice survived), while the control mice (MG or PBS) had a mortality rate of 16% (5 mice out of 6 survived in each batch).

In order to analyze the immune response, blood samples were taken in the retro-orbital sinus in the mice before they were sacrificed. The sera were recovered from these samples after they were centrifuged, and were stored at −80° C. until their specific IgG and IgA antibodies of the E7 antigen and various cytokines were analyzed.

In order to be able to determine the production of characteristic cytokines of a Th1-type immune response and to determine their cytotoxic activity in vitro, the splenocytes of the sacrificed mice were also isolated, then cultivated and restimulated with an E7 protein marked with a polyhistidine tag (His-tag), and were purified starting from Escherichia coli. For the cytotoxicity test, the restimulated splenocytes were transferred to a culture plate containing viable TC-1 cells, and incubated for 5 days.

Lysis of the TC-1 cells by the splenocytes was then observed under a microscope (FIG. 6, micrographs with 100× magnification). After 5 days of co-culture, the splenocytes of the mice immunized with the MG(pLB356) strain had clearly proliferated (FIG. 6C, white arrows), in contrast to the control treatments (FIGS. 6A and 6B). In addition, the TC-1 cells, which had formed a dense and quite adherent layer in the presence of the splenocytes of the control mice vaccinated with PBS (FIG. 6A) or with the MG control strain (FIG. 6B), almost completely disappeared in the presence of the splenocytes of the mice vaccinated with the MG(pLB356) recombinant strain (FIG. 6C, white circles), confirming that the splenocytes of these immunized mice induce very effective lysis of TC-1 tumor cells in vitro.

### EXAMPLE 6

### Use of IL-10-Producing Recombinant Bacteria to Treat Mice

Crohn's Disease and ulcerative colitis are the two principal forms of chronic inflammatory bowel disease (IBD). The inflammation observed in ulcerative colitis always affects the rectum and sometimes the colon. It does not in any case affect other segments of the digestive tract, although in the case of Crohn's Disease the inflammation can reach all parts of the digestive tract (from the mouth to the anus), but preferentially is located on the terminal portion of the small intestine (ileum) and on the colon.

The use of probiotics to treat these inflammatory diseases has been suggested for many years and various studies have shown the beneficial effect of such bacterial products tested alone or in combination. These results have suggested that probiotic bacteria may be used as carriers of a beneficial gene into the intestine, in addition to their intrinsic capabilities as anti-inflammatory agents. This strategy was used to express IL-10 in L. lactis bacteria under the control of the P1 promoter (Steidler et al., 2000, cited above). Clinical Phase I trials conducted with these recombinant bacteria have shown that their administration does not cause any deleterious effects in patients but produces only a short-term protective effect.

The MG(pLB350) recombinant strain showed a protective role in two different mouse models, in which acute colitis was caused by DSS (Dextran sulfate sodium) (model used by Steidler et al., 2000, cited above) or by trinitrobenzene sulfonic acid (TNBS) (model used by Foligne et al., Gastroenterology, 133(3): 862-74, 2007) (results not shown). In the present example, the MG(pLB350) recombinant strain is tested in a model of chronic colitis.

The model used reproduces the periods of recovery and recurrence observed in patients afflicted with Crohn's Disease. To do this, the colitis was induced in mice by intra-rectal injection of 2,4-dinitrobenzene sulfonic acid (DNBS). C57BL/6 male mice from 6 to 8 weeks old, distributed into 4 groups of 8 mice, first received (Day 1 to 3) a first dose of 2 mg of DNBS (groups 1 to 3) or ethanol (Group 4) as a control. A recovery period followed, between Day 4 and Day 14. Then, between Day 14 and Day 23, they were administered daily and orally 5×10⁹ CFU MG(pLB350) recombinant bacteria (Group 1), dexamethasone (Group 2), or PBS (groups 3 and 4). The dexamethasone is a glucocorticoid hormone used especially to treat various inflammatory diseases such as IBD. A second episode of inflammation was induced on Day 21 by administration of 1 mg of DNBS. The mice were sacrificed on Day 24.

The inflammation was evaluated according the Wallace criteria, and a Wallace score (grade from 0 to 10 based on the macroscopic lesions on the colon) was assigned to each mouse.

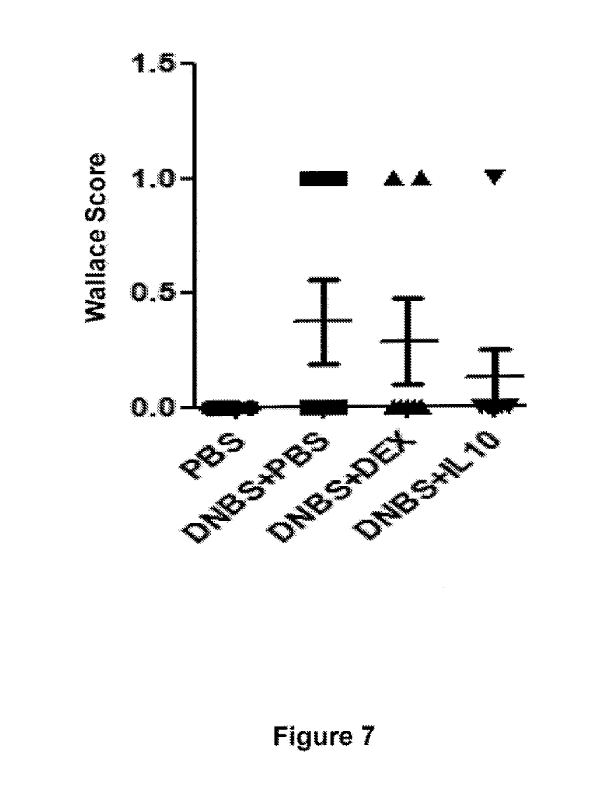

The results obtained (FIG. 7) demonstrate the protective effect of the MG(pLB350) strain following reactivation of the colitis by the second administration of DNBS (DNBS+IL10). This effect is greater than that observed following treatment with dexamethasone (DNBS+DEX).

1.-10. (canceled)

11. An expression cassette that includes:
(i) a promoter (PGroESL) of the GroESL operon of Lactococcus lactis, and;
(ii) a heterologous nucleotide sequence that codes for a polypeptide of interest, placed under the transcriptional control of said promoter.

12. The expression cassette according to claim 11 additionally including a nucleotide sequence that codes for an extracellular addressing peptide, under the transcriptional control of the promoter, said expression cassette enabling cloning of the heterologous nucleotide sequence in translational fusion with the said addressing peptide.

13. The expression cassette according to claim 12, wherein said extracellular addressing peptide is the signal peptide of the Exp4 protein of Lactococcus lactis, and has the following sequence:
(SEQ ID NO.: 5)
MKKINLALLTLATLMGVSSTAVVFA.

14. The expression cassette according to claim 11 wherein the heterologous nucleotide sequence codes for a therapeutic or vaccinal protein.

15. The expression cassette according to claim 14 wherein the heterologous nucleotide sequence codes for a protein selected from the group consisting of a cytokine; a vaccine antigen, of viral, bacterial or eukaryote origin; a protease inhibitor; a hormone; and an enzyme of bacterial or eukaryote origin.

16. The expression cassette according to claim 14 wherein the heterologous nucleotide sequence codes for a protein selected from the group consisting of interleukin-10; interleukin-12; interleukin-2; interleukin-4; Granulocyte-Macrophage Colony-Stimulating Factor; Transforming growth factor-beta; proteins E6, E7, and L1 of human papilloma virus (HPV) Type 16; the VapA protein of Rhodococcus equi; the L7/L12 protein of Brucella abortus; the Nuc protein of Staphylococcus aureus; Elafin (or Trappin-2); Secretory Leukocyte Protease Inhibitor; one of the serine protease inhibitors (serpin family); leptin; insulin; human growth hormone; Insulin-like Growth Factors; Epidermal Growth Factor; a catalase, a superoxide dismutase, a lipase, a polymerase, an isomerase, and a ligase.

17. A recombinant vector that includes an insert comprised of the expression cassette according to claim 11.

18. The recombinant vector of claim 17 wherein the expression cassette additionally includes a nucleotide sequence that codes for an extracellular addressing peptide, under the transcriptional control of the promoter, said expression cassette enabling cloning of the heterologous nucleotide sequence in translational fusion with the said addressing peptide.

19. The recombinant vector of claim 18, wherein said extracellular addressing peptide is the signal peptide of the Exp4 protein of Lactococcus lactis, and has the following sequence:
(SEQ ID NO.: 5)
MKKINLALLTLATLMGVSSTAVVFA.

20. A lactic acid bacterium of the genus Lactococcus, transformed by an expression cassette according to claim 11.

21. A lactic acid bacterium of the genus Lactococcus, transformed by an expression cassette according to claim 12.

22. A lactic acid bacterium of the genus Lactococcus, transformed by an expression cassette according to claim 13.

23. A lactic acid bacterium according to claim 20 which is a bacterium of the species Lactococcus lactis.

24. A medication or a vaccine which comprises a recombinant vector including an expression cassette according to claim 11 or a lactic acid bacterium transformed with said expression cassette, wherein the heterologous nucleotide sequence codes for a therapeutic or vaccinal protein.

25. The medication or vaccine according to claim 24 in which the heterologous nucleotide sequence codes for interleukin-10 or codes for the E7 antigen of human papillomavirus (HPV) Type 16.

26. A method for administration of a therapeutic or vaccinal protein to an individual in need of such administration which comprises administration of a recombinant vector comprising the expression cassette according to claim 11 wherein the heterologous nucleotide sequence codes for a therapeutic or vaccinal protein or administration of a lactic acid bacterium transformed by said expression cassette.

27. The method of claim 26 wherein a lactic acid bacterium is administered via a mucous membrane.

28. The method of claim 26 wherein the lactic acid bacterium is administered intranasally or orally.

29. The method according to claim 26, for treatment of chronic inflammatory bowel disease, in which the heterologous nucleotide sequence codes for interleukin-10.

30. The method according to claim 26, for administration of a vaccine against cervical cancer, wherein the heterologous nucleotide sequence codes for the E7 antigen of human papillomavirus (HPV) Type 16.


SEQUENCE LISTING (8 sequences)

SEQ ID NO: 1 - DNA, 82 bp, Lactococcus lactis ssp cremoris
  misc_binding (1)..(17): binding site CtsR
cttgactaaatctgaccattgagataaaataagaatatgttagcactcaactattaagagtgctaaaaataaaaaatggagg

SEQ ID NO: 2 - DNA, 82 bp, Lactococcus lactis ssp cremoris
cttgactaaatctgaccattgagataaaataagaatatgttagcacacaactattaagagtgctaaaaataaaaaatggagt

SEQ ID NO: 3 - DNA, 83 bp, Lactococcus lactis ssp lactis
cttgactaaatctgaccattgagataaaataagaatatgttagcactcgtttaataagagtgctaaaaaataaaaaatggagg

SEQ ID NO: 4 - DNA, 83 bp, Lactococcus lactis ssp lactis
cttgactaaatctgaccattgagataaaataagaatatgttagcactcgtttaacaagagtgctaaaaaataaaaaatggagg

SEQ ID NO: 5 - protein, 25 aa, Lactococcus lactis
MKKINLALLTLATLMGVSSTAVVFA

SEQ ID NO: 6 - DNA, 37 bp, Artificial sequence
  PCR sense primer
ccaagatctaaatgttttctcttgactaaatctgacc

SEQ ID NO: 7 - DNA, 40 bp, Artificial sequence
  PCR antisense primer
agctagcgttaataaagcaaggtttatttttttcatatac

SEQ ID NO: 8 - DNA, 105 bp, Lactococcus lactis
atctaaatgttttctcttgactaaatctgaccattgagataaaataagaatatgttagcactcaactattaagagtgctaaaaataaaaaatggaggaaagtata